SEQUENCE LISTING (3 sequences)

SEQ ID NO: 1 - protein, 13 aa, Artificial Sequence
  fragment of hexon 6 capsis protein
IAFRDNFIGLMYY

SEQ ID NO: 2 - protein, 13 aa, Artificial Sequence
  redutase motif + hexon 6 capsid protein
CHGCGGFIGLMYY

SEQ ID NO: 3 - protein, 13 aa, Artificial Sequence
  modified version of hexon 6 capsid protein
IAFRDNFIGLMYW

(19) United States
(12) Patent Application Publication
(10) Pub. No.: US 2016/0250255 A1
(43) Pub. Date: Sep. 1, 2016

(54) METHODS FOR INDUCTION OF ANTIGEN-SPECIFIC REGULATORY T CELLS

(71) Applicant: IMCYSE SA, Liège (BE)

(72) Inventors: Jean-Marie SAINT-REMY, Grez-Doiceau (BE); Vincent CARLIER, Enines (BE); Luc VANDER ELST, Obaix (BE)

(21) Appl. No.: 15/032,387

(22) PCT Filed: Oct. 29, 2014
(86) PCT No.: PCT/EP2014/073257

§ 371 Date: Apr. 27, 2016

(30) Foreign Application Priority Data
Oct. 30, 2013 (GB) 1319160.6

(51) Int. Cl. A61K 35/17; C12N 5/0783

(52) U.S. Cl. CPC A61K 35/17; C12N 5/0646; A61K 2035/124

(57) ABSTRACT

The present invention relates to methods of obtaining antigen-specific regulatory cells in vitro or in vivo. The regulatory cells are obtainable by inducing apoptosis of antigen-presenting cells by NKT cells. In particular, NKT cells are elicited, in vitro or in vivo, by exposure to CD1d-restricted NKT cell peptide epitopes either in natural configuration or modified as to contain a thioreductase motif within flanking residues. The present invention discloses methods to elicit immature antigen-presenting cells loaded with apoptotic cells or with apoptotic bodies for suppressing or preventing diseases such as autoimmune diseases, graft rejection and allergic diseases, and medicaments related thereto. Further disclosed are the use of antigen-specific regulatory cells for suppressing or preventing diseases such as autoimmune diseases, graft rejection and allergic diseases, and medicaments related thereto. Further disclosed are populations of antigen-specific regulatory cells obtained by this method.

### FIELD OF THE INVENTION

The present invention relates to a method of obtaining antigen-specific regulatory T cells and their use as a medicament to treat conditions such as autoimmune diseases, allergic diseases or graft rejection.

### BACKGROUND OF THE INVENTION

Regulatory T cells (Tregs), particularly regulatory T cells expressing the transcription repressor Foxp3, are essential in maintaining a normal immune homeostasis. In the absence of such cells, autoimmunity rapidly develops with clinical manifestations such as diabetes mellitus and other autoimmune diseases (reviewed in Sakaguchi et al. (2012) Nature Med. 18 54-58). Foxp3+ regulatory T cells are actively selected in the thymus and constitute the population of cells found in peripheral blood, which stably express Foxp3. The threshold at which cells are selected in the thymus upon cognate recognition of self-peptides presented by thymic epithelial cells is such that Foxp3+ cells with significant affinity are found in the peripheral blood, in contrast to effector T cells. A current view is that peripheral tolerance is maintained, inter alia, by a balance between autoantigen-specific effector cells with weak affinity and Foxp3+ cells with higher affinity, thereby providing equilibrium towards tolerance. In addition to this central selection of regulatory T cells, cells can be converted in the periphery to express the transcription factor Foxp3. However, expression is lower than in thymus-selected population and some reversibility of the acquired phenotype has been observed.

The properties of natural regulatory T cells, as selected in the thymus and characterized by high and stable Foxp3 expression, make them very attractive as a means to control pathologies characterized by auto-immune responses, as well as a therapeutic tool to keep unwanted responses to graft or to allergens under control, to cite just a few. However, the number of antigen-specific natural regulatory T cells in the periphery is very low and methods to expand them in vivo or even in vitro are neither well defined nor reliable. A method by which it would be possible to selectively expand population of regulatory T cells would carry the potential to prevent or suppress disease processes without affecting the overall capacity of the organism to mount beneficial responses.

Apoptosis, or programmed cell death, is a physiological mechanism which helps maintain tissue homeostasis (reviewed in Fuchs and Steller (2011) Cell 147, 742-758). It has been calculated that up to 10⁶ cells are destroyed by apoptosis every minute in a human body. The enormous amount of antigens liberated by cell death has to be kept under control so as to avoid eliciting immune response against self-proteins. In fact, apoptotic cells are taken up by scavenger cells, mainly immature dendritic cells, and then processed in a way to induce tolerance. Cross-presentation of antigens derived from apoptotic cells are presented in class II major histocompatibility complexes (MHC), which are known to elicit Foxp3+ Treg expansion. Thus, apoptosis of cells, which occur in the absence of inflammatory context, represent a physiological way by which regulatory T cells are expanded.

It is therefore desirable to devise a method by which it would be possible to induce apoptosis of cells presenting autoantigens or antigens to which an immune response is undesirable (such as, for example, in allergic diseases or graft rejection), which would then generate apoptotic bodies, leading to expansion of antigen-specific regulatory T cells. Non-specific immunosuppressive therapies known in the art generally lead to susceptibility to severe infections and other serious consequences, which negatively affect quality of life. As such, it would be advantageous to develop a method whereby antigen-specific regulatory T cells may be used to treat immune diseases without the undesirable effects of traditional therapies.

A general method has been described by which it is possible to elicit antigen-specific NKT cells by cognate recognition of CD1d-restricted NTK cell peptides epitopes (WO2012069572). Peptide-specific NKT cells were shown to induce apoptosis of the antigen-presenting cell by a CD1d-TCR interaction. Further, it has been described that CD1d-binding peptides show a significant increase of their capacity to elicit apoptosis of an antigen-presenting cell when a redox motif is added to the flanking residues of the peptide epitope (WO2012069568).

Kushwah et al. (2010) Eur. J. Immunol. 40, 1025-1035, describe the uptake of apoptotic cells by dendritic cells. These apoptotic cells were obtained in a non-specific way by UV irradiation, leading to a heterogeneous population of apoptotic cells.

Sag et al. (2014) J. Clin. Invest. 124, 3725-3740 describe a population of alphaGalCer treated NTK cells which acquire characteristics of regulatory cells, such as the production and secretion of Interleukin 10 (IL10), and the expression of proteins found on Tregs.

### SUMMARY OF THE INVENTION

The present invention provides methods for the expansion of regulatory T cells such as antigen-specific Foxp3 regulatory T cells by inducing apoptosis of antigen-presenting cells carrying CD1d-restricted NKT cell peptide epitopes derived from alloantigens released by a graft, from autoantigens or allergens.

NKT cells may be obtained by active immunization of an animal and prepared by affinity purification using magnetic beads coated with surface-specific antibodies. Alternatively, the NKT cells may be obtained in vitro, the method comprising the isolation of NKT cells from an animal and exposure in culture to CD1d-restricted epitopes. This epitope can be either in natural configuration or containing a thioreductase motif within flanking residues as described herein or in WO2012069572 and WO2012069568, respectively.

NKT cells may be used in vivo to induce apoptosis of antigen-presenting cells, the method comprising the transfer of NKT cells in an animal actively producing an immune response towards the antigen recognized by NKT cells.

NKT cells may be used in vitro in cultures with antigen-presenting cells presenting the epitope recognized by NKT cells to generate or obtain apoptotic bodies.

Apoptotic bodies obtained from in vitro cultures may be used to load immature antigen-presenting cells and the immature antigen-presenting cells may be used to generate or obtain antigen-specific regulatory T cells by cycles of stimulation using population of CD4+ T cells obtained from naïve animals.

Apoptotic bodies obtained from in vitro cultures may be used to load immature antigen-presenting cells and the immature antigen-presenting cells may be used to generate or obtain antigen-specific regulatory NKT cells by cycles of stimulation using population of CD4+ T cells obtained from naïve animals.

Immature antigen-presenting cells loaded with apoptotic bodies obtained from in vitro cultures may be used for passive transfer into an animal in need of treatment.

The immature antigen-presenting cells loaded with apoptotic bodies may be used in vitro to generate or obtain regulatory T cells, including class II restricted regulatory T cells and CD1d restricted NKT regulatory cells, which may then be used for passive transfer to an animal in need of treatment.

Regulatory T cells, including class II restricted regulatory T cells and CD1d restricted NKT regulatory cells, obtained (and/or isolated) by the methods described herein are used for the prevention or treatment of diseases in a subject in need for such a prevention or treatment. The disease can be an auto-immune disease, allergic disorder or graft rejection.

On aspect of the present invention relates to in vitro methods of obtaining antigen-specific regulatory T cells. These can be natural regulatory cells or induced regulatory cells. These methods comprise the steps of:

a) providing antigen-specific NKT cells for a proteic antigen, the antigen comprising an NKT cell peptide epitope, which peptide epitope is capable of binding to a CD1d molecule;
b) providing APCs presenting the antigen;
c) inducing apoptosis of the APCs of b) by exposing the APCs to the antigen-specific NKT cells of a);
d) isolating apoptotic cells and/or apoptotic bodies from the APCs which underwent apoptosis in step c;
e) incubating the apoptotic cells or the apoptotic bodies of step d) with cells capable of presenting antigens from the apoptotic cells or from the apoptotic bodies, thereby obtaining antigen presenting cells loaded with apoptotic cells or apoptotic bodies and;
f) contacting the loaded antigen presenting cells obtained in step e) with a source of CD4+ cells, thereby obtaining a population of antigen-specific regulatory cells. In embodiments of these methods the antigen comprising an NKT cell peptide epitope in step a) can be an antigen wherein the NKT cell peptide epitope, capable of binding to a CD1d molecule, occurs in the wild type sequence of the antigen; can be an antigen wherein the NKT cell peptide epitope, capable of binding to a CD1d molecule, is generated by mutagenesis of the sequence of the antigen, or can be an antigen wherein an NKT cell peptide epitope, capable of binding to a CD1d molecule, is attached to the antigen as a fusion protein.

In embodiments of these methods the antigen-specific NKT cells in step a) are obtained by contacting peripheral cells with a peptide comprising a CD1d-restricted NKT cell peptide epitope.

In specific embodiments the CD1d restricted NKT cell peptide epitope comprises the motif [WFYHT]-X-X-[ VILM]-X-X-[WFYHT], more specifically the motif [WF]-X-X-[IL]-X-X-[WF].

In specific embodiments the peptide further comprises a sequence with the motif C-X-X-[CTS] or [CST]-X-X-C, such as C-X(2)-C.

In embodiments of these methods the antigen-specific NKT cells in step a) are obtained from naïve CD4+ T cells or from polarized CD4+ T cells.

In embodiments of these methods the apoptotic cells or apoptotic bodies in step d are isolated by a method selected from the group consisting of affinity purification, centrifugation, gel filtration, magnetic beads sorting and fluorescence-activated sorting.

In embodiments of these methods the cells in step e) capable of presenting antigens from the apoptotic cells or from the apoptotic bodies are selected from the group consisting of dendritic cells, macrophages, B lymphocytes, cells capable of expressing MHC class II determinants, cells capable of expressing CD1d determinants.

In embodiments of these methods the cells in step e) capable of presenting antigens from the apoptotic cells or from the apoptotic bodies in step e) are selected from the group consisting of immature APCs obtainable by transformation of peripheral blood monocytes and bone-marrow derived precursors.

In specific embodiments the source of CD4+ cells in step f) are class II restricted CD4+ T cells.

In other specific embodiments the source of CD4+ cells in step are CD1d restricted CD4+ NKT cells.

In further embodiment the methods comprise a step of determining the expression of Foxp3 and CD4+ in the antigen-specific regulatory T cells.

In further embodiment the methods comprise a step of separating the antigen-specific regulatory T cells into distinct subsets based on the expression of surface markers, the production of cytokines or the expression of Foxp3.

Another aspect of the invention relates to populations of antigen-specific regulatory T cells, obtainable by the above described methods, such as populations of antigen-specific regulatory NKT cells.

Another aspect of the present invention relates to the use of populations of antigen-specific regulatory T cells, obtainable by the above described methods, for use as a medicament. For example in the treatment or prevention of an autoimmune disease, an allergic disease or a graft rejection (e.g. of cellular of tissue origin), the treatment or prevention of a systemic or an organ-specific autoimmune disease, or the treatment of a chronic inflammatory disease.

Examples of an autoimmune disease in this context are autoimmune diseases against an antigen selected from the group of antigens consisting of thyroglobulin, thyroid peroxidase, TSH receptor, insulin (proinsulin), glutamic acid decarboxylase (GAD), tyrosine phosphatase IA-2, myelin oligodendrocyte protein and heat-shock protein HSP65.

Examples of allergic diseases in the context of the present invention are allergic diseases against an allergen selected from the group consisting of an airborne allergen, a food allergen, a contact allergen and a systemic allergen.

### BRIEF DESCRIPTION OF THE DRAWINGS

FIG. 1 shows induction of apoptosis in dendritic cells loaded with no peptide, an unrelated non-cognate peptide or the cognate peptide of SEQ. ID NO: 1. The left hand side of the figure shows the percentage of apoptotic cells induced by addition of NKT cells expanded by exposure to peptide of SEQ. ID NO: 1; the right hand side shows the same results but obtained with NKT cells expanded with peptide of SEQ. ID NO: 2.

FIG. 2 presents data obtained in the same setting but using JAWS2 cells, which do not express MHC class II molecules.

FIG. 3 shows data obtained under the same conditions but with a B cell line (WEHI 231) from a MHC incompatible strain.

| Peptide sequences | |
|---|---|
| SEQ ID NO: 1 | IAFRDNFIGLMYY |
| SEQ ID NO: 2 | CHGCGGFIGLMYY |
| SEQ ID NO: 3 | IAFRDNFIGLMYW |

### DETAILED DESCRIPTION OF THE INVENTION

### Definitions

The term “peptide” as used herein refers to a molecule comprising an amino acid sequence of between 2 and 200 amino acids, connected by peptide bonds, but which can in a particular embodiment comprise non-amino acid structures (like for example a linking organic compound). Peptides according to the invention can contain any of the conventional 20 amino acids or modified versions thereof, or can contain non-naturally occurring amino acids incorporated by chemical peptide synthesis or by chemical or enzymatic modification.

In specific embodiments peptides have a length of at most 20, 25, 30, 50, 75, 100 or 150 amino acids.

Peptides comprising a NKT cell peptide epitope typically have a length of at least 7, 8, 9 or 10 amino acids.

Peptides comprising a NKT cell peptide epitope and a redox motif sequence typically have a length of at least 11, 12, 13, 14. Depending on the presence of additional amino acids between an NKT cell peptide epitope and a redox motif (0, 1, 2, 3, 4, 5, 6, 7 amino acids), such peptide has a length of any values between 11 to 18 amino acids. The term “antigen” as used herein refers to a structure of a macromolecule, typically protein (with or without polysaccharides) or made of proteic composition comprising one or more hapten(s) and comprising at least a CD1d-restricted NKT cell peptide epitope or a class II-restricted epitope.

The term “antigenic protein” as used herein refers to a protein comprising at least a CD1d-restricted NKT cell peptide epitope or a class II-restricted epitope. An auto-antigen or auto-antigenic protein as used herein refers to a human or animal protein present in the body, which elicits an immune response within the same human or animal body. Groups and specific examples of antigenic proteins are provided below in the section on diseases, where antigens are the causative agents of the mentioned disorders.

The term “food or pharmaceutical antigenic protein” refers to an antigenic protein naturally present in a food or pharmaceutical product, such as in a vaccine. Groups and specific examples of antigenic proteins are provided below in the section on diseases, where antigens are the causative agents of the mentioned disorders.

The term “epitope” refers to one or several portions (which may define a conformational epitope) of an antigenic protein which is/are specifically recognized and bound by an antibody or a portion thereof (Fab′, Fab2′, etc.) or a receptor presented at the cell surface of a B or T cell lymphocyte, and which is able, by this binding, to induce an immune response.

The term “T cell epitope” in the context of the present invention refers to a dominant, sub-dominant or minor T cell epitope, i.e. a part of an antigenic protein that is specifically recognized and bound by a receptor at the cell surface of a T lymphocyte. Whether an epitope is dominant, sub-dominant or minor depends on the immune reaction elicited against the epitope. Dominance depends on the frequency at which such epitopes are recognized by T cells and able to activate them, among all the possible T cell epitopes of a protein. In particular embodiments, a T cell epitope is an epitope recognized by MHC class II molecules, which consists of a sequence of +/−9 amino acids that fit in the groove of the MHC II molecule. Within a peptide sequence representing a T cell epitope, the amino acids in the epitope are numbered P1 to P9, amino acids N-terminal of the epitope are numbered P-1, P-2 and so on, amino acids C terminal of the epitope are numbered P+1, P+2 and so on.

More specifically it refers to epitopes as recognized by the immune system in humans.

“Redox motif”, also called “oxidoreductase motif” is a tetrapeptide motif with reducing activity with the sequence [CST]-X-X-C or C-X-X-[CST], as described further on in more detail.

The term “NKT cell peptide epitope” refers to a part of an antigenic protein that is specifically recognized and bound by a receptor at the cell surface of a T lymphocyte. In particular, a NKT cell peptide epitope is an epitope bound by CD1d molecules.

More specifically it refers to epitopes as recognized by the immune system in humans.

In the context of the present invention this relates to peptides which comprise in the CD1d binding part a sequence with the heptapeptide motif [FWYTH]-x-x-[VILM]-x-x-[FWYTH], as explained in more detail below.

The term “CD4+ effector cells” refers to cells belonging to the CD4-positive subset of T-cells whose function is to provide help to other cells, such as, for example B-cells. These effector cells are conventionally reported as Th cells (for T helper cells), with different subsets such as Th0, Th1, Th2, and Th17 cells.

The term “NKT cells” refers to cells of the innate immune system characterized by the fact that they carry receptors such as NK1.1 and NKG2D, and recognize epitopes presented by the CD1d molecule. In the context of the present invention, NKT cells can belong to either the type 1 (invariant) or the type 2 subset.

The “CD1d molecule” refers to a non-MHC derived molecule made of 3 alpha chains and an anti-parallel set of beta chains arranged into a deep hydrophobic groove opened on both sides and capable of presenting lipids, glycolipids or hydrophobic peptides to NKT cells.

The term “immune disorders” or “immune diseases” refers to diseases wherein a reaction of the immune system is responsible for or sustains a malfunction or non-physiological situation in an organism. Immune disorders in the context of the present invention refer to pathology induced by infectious agents and tumor surveillance.

The term “alloantigen” refers to an antigen generated by protein polymorphism in between 2 individuals of the same species.

The term “alloreactivity” refers to an immune response that is directed towards allelic differences between the graft recipient and the donor. Alloreactiity applies to antibodies and to T cells. The present invention relies entirely on T cell alloreactivity, which is based on T cell recognition of alloantigens presented in the context of MHC determinants as peptide-MHC complexes.

The term “major histocompatibility antigen” refers to molecules belonging to the HLA system in man (H2 in the mouse), which are divided in two general classes. MHC class I molecules are made of a single polymorphic chain containing 3 domains (alpha 1, 2 and 3), which associates with beta 2 microglobulin at the cell surface. Class I molecules are encoded by 3 loci, called A, B and C in humans. Such molecules present peptides to T lymphocytes of the CD8+ subset. Class II molecules are made of 2 polymorphic chains, each containing 2 chains (alpha 1 and 2, and beta 1 and 2). These class II molecules are encoded by 3 loci, DP, DQ and DR in man.

The term “minor histocompatibility antigen” refers to peptides that are derived from normal cellular proteins and are presented by MHC belonging to the class I and/or the class II complexes. Any genetic polymorphism that qualitatively or quantitatively affects the display of such peptides at the cell surface can give rise to a minor histocompatibility antigen.

The term “homologue” as used herein with reference to the epitopes used in the context of the invention, refer to molecules having at least 50%, at least 70%, at least 80%, at least 90%, at least 95% or at least 98% amino acid sequence identity with the naturally occurring epitope, thereby maintaining the ability of the epitope to bind an antibody or cell surface receptor of a B and/or T cell. Particular embodiments of homologues of an epitope correspond to the natural epitope modified in at most three, more particularly in at most 2, most particularly in one amino acid.

The term “derivative” as used herein with reference to the peptides of the invention refers to molecules which contain at least the peptide active portion (i.e. capable of eliciting cytolytic CD4+ NKT cell activity) and, in addition thereto comprises a complementary portion which can have different purposes such as stabilizing the peptides or altering the pharmacokinetic or pharmacodynamic properties of the peptide.

The term “sequence identity” of two sequences as used herein relates to the number of positions with identical nucleotides or amino acids divided by the number of nucleotides or amino acids in the shorter of the sequences, when the two sequences are aligned. In particular embodiments, this sequence identity is from 70% to 80%, from 81% to 85%, from 86% to 90%, from 91% to 95%, from 96% to 100%, or 100%.

The terms “peptide-encoding polynucleotide (or nucleic acid)” and “polynucleotide (or nucleic acid) encoding peptide” as used herein refer to a nucleotide sequence, which, when expressed in an appropriate environment, results in the generation of the relevant peptide sequence or a derivative or homologue thereof. Such polynucleotides or nucleic acids include the normal sequences encoding the peptide, as well as derivatives and fragments of these nucleic acids capable of expressing a peptide with the required activity. According to one embodiment, the nucleic acid encoding the peptides according to the invention or fragment thereof is a sequence encoding the peptide or fragment thereof originating from a mammal or corresponding to a mammalian, most particularly a human peptide fragment.

The term “organic compound having a reducing activity” refers in the context of this invention to compounds, more in particular amino acid sequences, with a reducing activity for disulfide bonds on proteins.

The term “immune disorders” or “immune diseases” refers to diseases wherein a reaction of the immune system is responsible for or sustains a malfunction or non-physiological situation in an organism. Included in immune disorders are, inter alia, allergic disorders and autoimmune diseases.

The terms “allergic diseases” or “allergic disorders” as used herein refer to diseases characterized by hypersensitivity reactions of the immune system to specific substances called allergens (such as pollen, stings, drugs, or food). Allergy is the ensemble of signs and symptoms observed whenever an atopic individual patient encounters an allergen to which he has been sensitized, which may result in the development of various diseases, in particular respiratory diseases and symptoms such as bronchial asthma. Various types of classifications exist and mostly allergic disorders have different names depending upon where in the mammalian body it occurs.

“Hypersensitivity” is an undesirable (damaging, discomfort-producing and sometimes fatal) reaction produced in an individual upon exposure to an antigen to which it has become sensitized; “immediate hypersensitivity” depends of the production of IgE antibodies and is therefore equivalent to allergy.

The terms “autoimmune disease” or “autoimmune disorder” refer to diseases that result from an aberrant immune response of an organism against its own cells and tissues due to a failure of the organism to recognize its own constituent parts (down to the sub-molecular level) as “self”. The group of diseases can be divided in two categories, organ-specific (such as Addison disease, hemolytic or pernicious anemia, Goodpasture syndrome, Graves disease, idiopathic thrombocytopenic purpura, insulin-dependent diabetes mellitus, juvenile diabetes, uveitis, Crohn's disease, ulcerative colitis, pemphigus, atopic dermatitis, autoimmune hepatitis, primary biliary cirrhosis, autoimmune pneumonitis, auto-immune carditis, myasthenia gravis, glomerulonephritis and spontaneous infertility) and systemic diseases such as lupus erythematosus, psoriasis, vasculitis, polymyositis, scleroderma, multiple sclerosis, ankylosing spondilytis, rheumatoid arthritis and Sjoegren syndrome). The autoimmune disorders are thus directed to own cells or tissues and include a reaction to “auto-antigens”, meaning antigens (e.g. of proteins) that are own constituent parts of the specific mammalian organism. In this mechanism, auto-antigens are recognised by B- and/or T-cells which will install an immune reaction against such auto-antigen.

A non-limitative list of diseases encompassed by the term “auto-immune diseases” or “auto-immune disorders” comprises therefore Acute disseminated encephalomyelitis (ADEM), Addison's disease, Alopecia areata, Antiphospholipid antibody syndrome (APS), Autoimmune hemolytic anemia, Autoimmune hepatitis, Bullous pemphigoid, Behçet's disease, Coeliac disease, inflammatory bowel disease (IBD) (such as Crohns Disease and Ulcerative Colitis), Dermatomyositis, Diabetes mellitus type 1, Goodpasture's syndrome, Graves' disease, Guillain-Barré syndrome (GBS), Hashimoto's disease, Idiopathic thrombocytopenic purpura, Lupus erythematosus, Mixed Connective Tissue Disease, Multiple sclerosis (MS), Myasthenia gravis, Narcolepsy, Pemphigus vulgaris, Pernicious anaemia, Psoriasis, Psoriatic Arthritis, Polymyositis, Primary biliary cirrhosis, Rheumatoid arthritis (RA), Sjögren's syndrome, Temporal arteritis, Vasculitis, Wegener's granulomatosis and atopic dermatitis.

An “allergen” is defined as a substance, usually a macromolecule or a proteic composition which elicits the production of IgE antibodies in predisposed, particularly genetically disposed, individuals (atopics) patients. Similar definitions are presented in Liebers et al. (1996) Clin. Exp. Allergy 26, 494-516.

The term “inflammatory diseases” or “inflammatory disorders” refers to diseases wherein the typical characteristics of inflammation are observed. This term can therefore overlap with other diseases wherein an inflammation aspect is also present.

It is known in the art that a distinction can be made between “acute inflammation” and “chronic inflammatory diseases”. The term “inflammatory diseases” or “inflammatory disorders” includes but is not limited to disease selected from the group of rheumatoid arthritis, conjunctivitis, rheumatoid spondylitis, osteoarthritis, gouty arthritis, bronchitis, tuberculosis, chronic cholecystitis, inflammatory bowel disease, acute pancreatitis, sepsis, asthma, chronic obstructive pulmonary disease, dermal inflammatory disorders such as psoriasis and atopic dermatitis, systemic inflammatory response syndrome (SIRS), acute respiratory distress syndrome (ARDS), cancer-associated inflammation, reduction of tumor-associated angiogenesis, diabetes, treatment of graft v. host disease and associated tissue rejection inflammatory responses, Crohn's disease, delayed-type hypersensitivity, immune-mediated and inflammatory elements of CNS disease; e.g., Alzheimer's, Parkinson's, multiple sclerosis, etc.

The term “therapeutically effective amount” refers to a number of cells which produces the desired therapeutic or preventive effect in a patient. For example, in reference to a disease or disorder, it is the number of cells which reduces to some extent one or more symptoms of the disease or disorder, and more particularly returns to normal, either partially or completely, the physiological or biochemical parameters associated with or causative of the disease or disorder. According to one particular embodiment of the present invention, the therapeutically effective number is the number of cells which will lead to an improvement or restoration of the normal physiological situation. For instance, when used to therapeutically treat a mammal affected by an immune disorder, it is a daily number of cells per kg body weight of this mammal.

The term “natural” when referring to a peptide or a sequence herein relates to the fact that the sequence is identical to a naturally occurring sequence. In contrast therewith the term “artificial” refers to a sequence or peptide which as such does not occur in nature. Optionally, an artificial sequence is obtained from a natural sequence by limited modifications such as changing one or more amino acids within the naturally occurring sequence or by adding amino acids N- or C-terminally of a naturally occurring sequence. Amino acids are referred to herein with their full name, their three-letter abbreviation or their one letter abbreviation.

“Regulatory T cells” are defined as cells exerting a suppressive activity on the activation of effector cells. Effector cells can be class II restricted CD4+ T cells, class I restricted CD8+ T cells, antigen presenting cells, NKT cells or Natural Killer (NK) cells. This description encompasses the classical “class II restricted regulatory T cells” (aka as “Foxp3+ regulatory T cells” or “Foxp3+CD4+CD25+ regulatory T cells”), as well as NKT regulatory cells (NKT regs). The NKT regulatory cells are CD1d restricted and further optionally defined by the expression of the transcription factor “Promyelocytic Leukaemia Zinc Finger protein” (PLZF).

“NKT regulatory cells” are thus cells equally excerting a suppressive activity on the above mentioned effector cells.

### DETAILED DESCRIPTION OF THE INVENTION

The general principle of the present invention is to use apoptotic bodies obtained from specific antigen-loaded antigen-presenting cells to elicit the production of antigen-specific regulatory T cells, such as Foxp3+ regulatory T cells and CD1 d restricted NKT regulatory cells. Specifically, the purpose of the present invention is to provide a method by which selective apoptosis is obtained from antigen-presenting cells presenting an autoantigen or an antigen to which an immune response is undesirable (e.g. an allergen, an alloantigen from a graft or an allofactor used of therapeutic purposes), in the context of CD1d determinants. As described in greater detail below, the present invention therefore provides a method of generating, obtaining or isolating antigen-specific regulatory T cells, including class II restricted regulatory T cells and CD1d restricted NKT regulatory cells, thereby providing the possibility of switching off an immune response specific for a given antigen.

Cells induced in apoptosis proceed through a number of surface alterations, including oxidation of phosphatidylserine, polysaccharides, and glycolipids, which render them recognizable by phagocytes. In addition, apoptotic cells express new proteins, such as thrombospondin-1 and/or localize at their surface intracellular components such as phosphatidylserine, DNA and nucleosomes. Altogether these surface alterations provide a possibility for phagocytes to engulf apoptotic cells without triggering an innate immune reaction, in the absence of ligation by innate receptors such as Toll-like receptors, NOD or RIG.

Phagocytic cells removing apoptotic cells are equipped with recognition receptors, such as CD14, scavenger receptors and C-type lectin receptors (Ravichandran & Lorenz, (2007) Nature Rev. Immunol. 7, 964-974). Scavenger receptors are surface glycoproteins able to bind oxidized or acetylated low-density lipoproteins (LDL) as well as polyanionic ligands and apoptotic cells. Examples of scavenger receptors include CD36, LOX-1 and CLA-1. Recognition is followed by rapid internalization and, in the case of apoptotic cells, destruction and fusion with endosomes and lysosomes. Of particular interest is the production of thrombospondin-1 by apoptotic cells, which acts as a soluble bridge with CD36 expressed on phagocytes. Expression of thrombospondin-1 is caspase-dependent.

Soluble factors also play a role in the removal of apoptotic cells. Examples include collectins and collectin-like molecules, such as mannose binding lectin and C1q. Both interact with calreticulin expressed at the surface of phagocytes. The family of pentraxins, which include serum amyloid P (SAP) and C-reactive protein (CRP) and prototypic pentraxin (PTX) also bind to apoptotic cells.

Altogether, there are a large number of factors that are used under physiological conditions to dispose of cells undergoing natural programmed cell death, or apoptosis (Jeannin et al. (2008) Curr. Opinion Immunol. 20, 530-537). In mammals, there is a constant renewal of cells to maintain normal cell numbers and activity (Steinman et al. (2000) J. Exp. Medicine 191, 411-416). In the absence of inflammatory conditions, apoptotic bodies are taken up in organs by antigen-presenting cells, which migrate towards regional lymph nodes in which an exchange of apoptotic bodies occurs with lymph node dendritic cells, either directly or as a consequence of the rapid lysis of the migrating antigen-presenting cells. At least some of the dendritic cells migrating to regional lymph nodes are found to be immature, which exhibit a high capacity to phagocyte apoptotic cells. In the lymph node, dendritic cells are primarily in an immature status, but seemingly belong to a subset showing the capacity to present antigens in both class I and class II determinants. In the absence of co-stimulatory signals related to the non-inflammatory conditions, MHC class II presentation provides the recruitment and activation signals required for regulatory T cells. Such regulatory T cells are antigen-specific (directed towards autoantigens) and suppress activation of a response towards such autoantigens. Thus, apoptosis occurring in a non-inflammatory context elicits antigen-specific regulatory T cells, which maintain tolerance to self-antigens.

Conversely, under inflammatory conditions, as it occurs in autoimmune diseases or responses to alloantigens or allergens or during an immune response elaborated against infectious agents, there is an increase in the production of apoptotic bodies, which are carried to regional lymph nodes. This massive influx of cells loaded with apoptotic bodies exceeds the capacity of lymph node dendritic cells to capture such bodies to present them in order to recruit and activate regulatory T cells. Additionally, the presence of pro-inflammatory cytokines alters the phenotype of lymph node dendritic cells, which are induced into maturation and, consequently, increases activation of effector T cells to the detriment of regulatory T cells. Although this is a desirable effect during a response to infectious agents, in the context of autoimmune diseases, allergic reactions, and graft rejection, it unfortunately leads to further tissue destruction and inflammation. It would therefore be advantageous to devise a novel method to increase the capacity to generate or obtain apoptotic bodies in a non-inflammatory context to generate, obtain or isolate antigen-specific regulatory T cells.

During studies on the elicitation of cytotoxic cells using T cell epitopes in natural configuration or carrying a thioreductase motif within flanking residues, it was unexpectedly found that a consequence of the induction of cytotoxic cells was an accumulation of Foxp3+ regulatory T cells in target organs. Thus, in a model of skin graft rejection, the long-term persistence of an allogeneic graft was accompanied by the presence of Foxp3+ regulatory T cells in the graft itself. The same observation was made in experimental models of multiple sclerosis, in which prevention or suppression of diseases was accompanied by accumulation of Foxp3+ regulatory T cells in central nervous system (CNS) white matter. The present findings therefore provide the rationale and teaching to practice the invention

The present invention therefore also comprises in a particular embodiment the use of CD1d-restricted NKT cell peptide epitopes, optionally with an additional thioreductase motif within flanking residues as described in WO2012069572 and WO2012069568, respectively (which are included herein by reference).

It should be understood that NKT cells elicited by exposure to a CD1d-presented peptidic epitope have an intrinsic property to induce apoptosis of the antigen-presenting cell with which a synapse is formed between the CD1d-bound peptide complex and the antigen-specific receptor of NKT cells. However, addition of a thioreductase motif within the flanking residues of the CD1-bound peptide maintains the capacity of NKT cells to induce apoptosis, or depending or the experimental conditions may even increase this capacity.

In general, the peptides to be used in the present invention are peptides which comprise at least one T-cell epitope of an antigen (self or non-self) with a potential to trigger an immune reaction. These peptides can be used without altering their natural sequence, or after addition, substitution or deletion of amino acids so as to increase the interaction with the Cd1d molecule (as described in WO2012069572), or after addition of a thioreducatase motif within the epitope flanking residues (as described in WO2012069568). It should further be understood that such peptides can be the result of any combination of addition, deletion or substitution of amino acids and addition of a thioreductase motif within flanking residues.

The thioreductase motif is an organic compound having a reducing activity, such as a thioreductase sequence motif [CST]-X(2)-[CST]. The NKT cell epitope and the organic compound are optionally separated by a linker sequence. In particular embodiments, peptides to be used in the present invention comprise the thioreductase sequence motif [CST]-X(2)-[CST] wherein at least one of [CST] is Cys; thus the motif is either C-X(2)-[CST] or [CST]-X(2)-C. In particular embodiments peptides of the invention contain the sequence motif C-X(2)-[CS] or [CS]-X(2)-C. In more particular embodiments peptides contain the sequence motif C-X(2)-S, S-X(2)-C or C-X(2)-C. In the above section, and in other parts of the application square brackets [ ] are used to indicate alternative amino acids at one position in a peptide. Round brackets ( ) within a number indicate a repeat. (X)2 means thus X-X.

These peptides can be made by chemical synthesis, which allows the incorporation of non-natural amino acids. Accordingly, in the motif of reducing compounds according to particular embodiments of the present invention, C represents either cysteine or another amino acids with a thiol group such as mercaptovaline, homocysteine or other natural or non-natural amino acids with a thiol function. In order to have reducing activity, the cysteines present in the motif should not occur as part of a cystine disulfide bridge. Nevertheless, the motif may comprise modified cysteines such as methylated cysteine, which is converted into cysteine with free thiol groups in vivo.

The amino acid X in the [CST]-X(2)-[CST] motif of particular embodiments of the reducing compounds of the invention can be any natural amino acid, including S, C, or T or can be a non-natural amino acid. In particular embodiments X is an amino acid with a small side chain such as Gly, Ala, Ser or Thr. In further particular embodiments, X is not an amino acid with a bulky side chain such as Tyr. In further particular embodiments at least one X in the [CST]-X(2)-[CST] motif is His or Pro. In yet further embodiments X is not C.

In the present invention, the above described compound redox motif, if present, is placed either immediately adjacent to the NKT cell peptide epitope sequence within the peptide, or is separated from the NKT cell peptide epitope by a linker. More particularly, the linker comprises an amino acid sequence of 7 amino acids or less. Most particularly, the linker comprises 1, 2, 3, or 4 amino acids. Alternatively, a linker may comprise 6, 8 or 10 amino acids. In those particular embodiments of the peptides of the invention where the motif sequence is adjacent to the epitope sequence this is indicated as position P-4 to P-1 or P+1 to P+4 compared to the epitope sequence.

An NKT cell peptide epitope in a protein sequence can be identified by functional assays and/or one or more in silico prediction assays. The amino acids in a NKT cell peptide epitope sequence are numbered according to their position in the binding groove of the MHC proteins. In particular embodiments, the NKT cell peptide epitope present within the peptides of the invention consists of between 8 and 25 amino acids, yet more particularly of between 8 and 16 amino acids, yet most particularly consists of 8, 9, 10, 11, 12, 13, 14, 15 or 16 amino acids. In a more particular embodiment, the NKT cell peptide epitope consists of a sequence of 9 amino acids. In a further particular embodiment, the NKT-cell epitope is an epitope, which is presented to NKT cells by CD1d molecules. In particular embodiments of the present invention, the NKT cell peptide epitope sequence is an epitope sequence which fits into the cleft of a CD1d protein, more particularly a 7-amino acid peptide fitting into the CD1d cleft. This the heptapeptide has the general motif [FWYTH]-x-x-[VILM]-x-x-[FWYTH]. The amino acids in this peptide are numbered P1 to P7. In a more narrow version of this motif the amino acids P1 and/or P7 is [FWYH], [FWH] or [FW]. In further alternatives the amino acid at P4 is I or L. A specific version of the motif is [FW]-x-x-[IL]-x-x-[FW].

The NKT cell peptide epitope of the peptides of the present invention can correspond either to a natural epitope sequence of a protein or can be a modified version thereof, provided the modified NKT cell peptide epitope retains its ability to bind within the CD1d cleft, similar to the natural NKT cell peptide epitope sequence. The modified NKT cell peptide epitope can have the same binding affinity for the CD1d protein as the natural epitope, but can also have a lowered affinity. In particular embodiments the binding affinity of the modified peptide is no less than 10-fold less than the original peptide, more particularly no less than 5 times less.

If a natural NKT cell epitope is present in an antigen which comprises a class II restricted epitope, it is sufficient to use the existing NKT cell peptide epitope sequence to induce or increase the apoptotic properties of the NKT cells. Apoptotic bodies are engulfed and taken up by immature dendritic cells for presentation via class II MHC determinants which are present in the antigen.

If the NKT cell peptide epitope is obtained by mutagenesis or provided as a fusion protein, typically the whole antigen sequence is used, such that the antigen contains both a CD1d restricted NKT cell peptide epitope and one or more class II restricted epitopes.

Alternatively, it is possible to use a fragment of the modified antigen, as long as, apart from the introduced CD1 d restricted NKT cell peptide epitope, there is at least one MHC class II restricted epitope present in this fragment.

Examples of (auto-)antigens from which the NKT cell peptide epitopes can be derived for use in the invention are glutamic acid decarboxylase (GAD), myelin oligodendrocyte protein (MOG), the nicotinic muscle acetylcholine receptor and alpha gliadin.

Without intending to be limiting, the general mechanism of action of the present invention is as follows:
(a) Immature dendritic cells loaded with apoptotic bodies elicit regulatory T cells specific for determinants presented in MHC class II determinants,
(b) The regulatory T cells migrate to the location in which there is an unwanted immune response,
(c) The accumulation of the regulatory T cells in the target location results in a control of inflammation as a consequence of the numerous anti-inflammatory properties of the regulatory T cells,
(d) Tissue destruction and the production of apoptotic cells is suppressed and normal cell turnover in the target location is re-established thereby restoring normal tissue function.

A similar process is envisaged wherein immature dendritic cells loaded with apoptotic bodies elicit regulatory T cells specific for determinants presented in CD1d determinants.

The present invention provides various embodiments by which antigen-specific regulatory T cells can be obtained. In an embodiment of the invention, apoptosis of antigen-presenting cells may be obtained in vitro by exposure to NKT cells. Apoptotic bodies are used to load immature dendritic cells. The immature dendritic cells loaded with apoptotic bodies may then either be used for cell therapy or used in vitro for generating, isolating or obtaining antigen-specific regulatory T cells, which may then be used for cell therapy. Cell therapy in the context of the present invention comprises the step of preparing cells for administration to a mammal.

A general method for inducing of apoptosis of cells in vitro is described and known in the art. For example, apoptosis of CD4+ T cells lymphocytes can be obtained by culturing them in the presence of insolubilized antibodies to CD3 and CD28. The methods used to determine whether cells are actually apoptotic are well described in the art. Such methods include the binding of annexin V on phospholipids expressed at the surface of apoptotic cells, activation of caspases, and degradation of nucleic acid. Reviews on these methods can be found in publications such as in Fuchs and Steller (2011) Cell 147, 742-758.

In the present invention, and unlike the prior art, apoptosis induced in antigen-presenting cells requires the formation of a synapse between the antigen-presenting cell (APC) and the cell inducing apoptosis, i.e. NKT cells. The formation of a synapse activates the cytolytic properties of the NKT cell, resulting in induction of apoptosis only of the cells presenting the corresponding antigen-derived CD1 d restricted NKT cell peptide epitopes. Advantageously, this provides strict antigen specificity. In the absence of any additional reagent for the assay system, such as anti-CD3 antibodies, the in vitro induction of apoptosis described in the present invention reproduces conditions close to those occurring in vivo.

Apoptosis of antigen-presenting cells by CD4+ T cells has been reported by Janssens et al. (2003) J. Immunol. 171, 4604-4612). Regulatory T cells, under some circumstances, could induce target cell apoptosis and a number of mechanisms of induction have been described, including activation of IDO release of granzyme B with or without perforin. A review of these mechanisms can be found in (Shevach (2011) Adv. Immunol. 112, 137-176). In the present invention, the NKT cells represent a unique cell subset, distinct from regulatory T cells on both phenotypic and functional properties. The methods by which such NKT cells can be induced can be found in WO2012069572 and WO2012069568.

Methods for the identification and isolation of apoptotic bodies are known in the art. Apoptotic cells or apoptotic bodies express a number of novel constituents at their surface and can, in addition, be opsonized by soluble factors, as described above. These two types of alterations provide ways to isolate apoptotic cells or apoptotic bodies. Examples of this can be found in the art (Schiller et al. (2008) Cell Death Diff. 15, 183-191). One example is the use of an antibody to thrombospondin to isolate cells or cell debris, which, because of entering into an apoptotic cycles, express thrombospondin.

In an embodiment of the present invention, isolated apoptotic bodies or apoptotic cells are incubated with dendritic cells to allow engulfment, processing, and presentation in the context of MHC class II determinants. Different subsets of dendritic cells have been described, varying in function, surface phenotype, and maturity. In general, an immature dendritic cell has a high capacity to take up apoptotic cells and apoptotic bodies, but may not be efficient in terms of expression of epitopes within MHC class II determinants. However, some subsets of dendritic cells, in particular those housed within lymph nodes, combine the two properties, uptake of apoptotic cells or apoptotic bodies and presentation of epitopes at their surface.

In the context of the present invention, however, dendritic cells are derived in vitro and kept immature by methods well described in the art. The prior art teaches that derivatization of dendritic cells in the presence of interferon-gamma (IFN-gamma) induces a highly mature status, whereas IL-4 will maintain dendritic cells in an immature status. Dendritic cells can be derived from either peripheral blood monocytes or from bone marrow precursors. Apoptotic cells and apoptotic bodies obtained as described above are incubated with immature dendritic cells, thereby allowing presentation by MHC class II determinants.

It should be clear to one skilled in the art that dendritic cells are a preferred, but not exclusive means to obtain presenting cells capable of presenting antigens processed from apoptotic cells or apoptotic bodies. Alternatives include but are not limited to macrophages, endothelial, or epithelial cells, which can be induced in MHC class II expression.

In another embodiment of the present invention, dendritic cells loaded with antigens derived from apoptotic cells or apoptotic bodies may be used for cell therapy. By way of example, dendritic cells presenting class II restricted epitopes derived from apoptotic bodies obtained by the cytolytic action of NKT cells on antigen-presenting cells presenting an autoantigen may be administered intravenously to animals affected by a disease process in which an immune response to the autoantigen is implicated. The result of such cell therapy is the specific suppression of the immune response and the cure of the disease. Additional examples are provided below, but the scope of the present invention is not restricted to such examples.

In another embodiment, immature dendritic cells loaded with apoptotic cells or with apoptotic bodies are maintained in culture to which a population of CD4+ T cells is added for incubation to generate, isolate or obtain regulatory T cells.

Several possible sources of CD4+ T cells can be used in this embodiment, including but not limited to: 1) cells obtained from naïve animals and prepared by affinity using, for instance, magnetic beads coated with specific antibodies, including specific affinity separation of class II restricted CD4+ cells or of CD1 d restricted CD4+ cells; 2) polarized CD4+ T cells obtained from the spleen, lymph nodes, tissues, or peripheral blood from animals in which a disease process is ongoing related to an immune response to the (auto)antigen to which it is desirable to elicit regulatory T cells; or 3) natural regulatory T cells, as defined as showing high and stable expression of the Foxp3 repressor of transcription

It should be clear to one skilled in the art that each one of these 3 sources of CD4+ T cells may be more appropriate than the others, given relevant circumstances. By way of example, naïve CD4+ T cells are easily accessible even from peripheral blood and provide a repertoire, which is large enough to recognize any antigen. In situations in which it is preferred to prevent a disease process, and thereby in which the antigen can be chosen according, inter alia, to the MHC class II haplotype of a given animal, naïve CD4+ T cells would represent the best choice. On the other hand, polarized CD4+ T cells represent a source of cells for the practice of the present invention in situations in which it is preferred to use cells with increased affinity for peptide-MHC complexes. One example is provided by autoreactive CD4+ T cells found in type 1 diabetes, in which the recognition of insulin-derived peptides by CD4+ T cells occurs primarily through incomplete binding to peptide-MHC complexes, resulting in a relatively low T cell receptor affinity.

In the present invention, one preferred embodiment is to use natural regulatory T cells as a source. The repertoire of regulatory T cells is shaped towards recognition of self-antigens and, as described above, such cells have a sufficient affinity to functionally form synapse with antigen-presenting cells. The number of antigen-specific natural regulatory T cells towards a given antigen is exceedingly low as such represent only 5 to 10% of the total CD4+ T cell number. The present invention provides a method by which such low numbers can be increased in vitro. A further advantage of using natural regulatory T cells regulatory cells in the present invention is their reported phenotypic stability. Thus, in natural, expression of Foxp3 is high and remains stable over time and under various activation conditions. By contrast, regulatory T cells induced into the periphery and acquiring Foxp3 expression may be unstable and loose their regulatory properties when the context changes in which they are active, as for instance under inflammatory conditions.

In present invention, it is shown that antigen presenting cells, such as iDC, which are loaded with apoptotic cells, can convert a cell with a CD4+ phenotype into a cell with regulatory properties.

It has recently emerged that NKT cells, which are equally CD4+, can convert into a cell type with regulatory properties, including the production of IL-10 (Sag et al. cited above). The examples section of the present application provides support for a parallel between the induction of class II restricted CD4+ T cell and the induction of CD1 d restricted NKT cell into a regulatory T cells.

Contrary to the prior art on NKT cells which are typically induced by a physiologically irrelevant glycolipid such as alphaGalCer, the experiments of the present invention are performed with peptide epitopes which bind to the CD1d molecule and mimic physiological relevant processes.

It is an aspect of the present invention that APC, loaded with apoptotic bodies, can induce CD4+ T cells into a cell with a regulatory phenotype and that this methodology can be applied on class II restricted CD4+ T cell to obtain at a classical foxp3 CD4+ regulatory T cell, but can also be applied on a CD1d restricted CD4+ NKT cell to obtain a NKT cell with regulatory properties optionally further defined by the cell markers disclosed in Example 9.

In another embodiment of the invention, regulatory T cells expanded (natural regulatory T cells) or induced (naïve or polarized) by in vitro culture with immature dendritic cells presenting antigens derived from apoptotic cells or apoptotic bodies are used for cell therapy. Such therapy can be administered as a preventive therapy, as for example in the prevention of graft rejection, or as a suppressive therapy, as for instance in type 1 diabetes.

Optionally, regulatory T cells obtained by methods described in the present invention can be further expanded using non-specific means when it is desired to further increase the number of such regulatory T cells. Examples of such non-specific methods are known in the art. For instance, cells incubated in the presence of insolubilized anti-CD3 and anti-CD28 antibodies and IL-2 can be expanded by several orders of magnitude.

It should be clear to one skilled in the art that, prior to cell administration, further steps could be added. One possibility is to further restrict the specificity of regulatory T cells by incubating cells with tetramers of MHC class II determinants loaded with one or more of a synthetic peptide to which it is desirable to orientate regulatory T cells. Another possibility is to sort out cells by a surface marker or various degree of Foxp3 expression. A population of cells with particularly high expression of Foxp3 is known to be part of the whole natural regulatory T cell population and present characteristics which make them particularly suitable in the context of the present invention.

In another embodiment, regulatory T cells obtained by the present invention can be used to establish the relevance of a given antigen or epitope for the development of a disease process. In many diseases, there is more than one antigen involved in the process, yet it remains difficult to identify the most important one. Producing antigen-specific regulatory T cells by practicing the present invention provides a method to switch off specific antigens as a means to isolate and identify the role of specific antigens in the development of disease.

In another embodiment, antigen-specific regulatory T cells obtained by the present invention provides a method to determine the importance of the regulatory T cell phenotype in its function. As an example, antigen-specific regulatory T cells may be sorted according to expression of granzyme and populations of granzyme+ and granzyme(−) are compared in terms of capacity to suppress a response either in vitro or in vivo. Yet another example is the expression of a surface marker such as neuropilin.

The various applications of the present invention are illustrated in the following examples. There is, however, no intention to restrict the scope of the invention to such exam pies.

### EXAMPLES

### Example 1

### Induction of Apoptosis In Vitro

Gene therapy and gene vaccination using viral vectors cannot be practiced due to a strong immune response elicited in recipients of gene therapy or gene vaccination towards proteins of the viral vector backbone wherein a therapeutic gene is cloned. It would therefore be advantageous to suppress such response against the viral protein of the backbone of the viral vector, thereby allowing long-term expression of the transgene or strong immunogenicity due to the persistence of the immunogen. The present example illustrates the use of NKT cells specific for antigenic determinants of the viral capsid to eliminate antigen-presenting cells presenting CD1d-bound epitopes.

Antigen-presenting cells (APC) are prepared from C57BL/6 mice and loaded with a peptide encompassing a CD1d-restricted NKT cell peptide epitope of hexon-6 capsid protein of adenovirus 5 vector used for gene therapy or gene vaccination.

Thus, the following peptides are used:

IAFRDN FIGLMYY [SEQ. NO: 1], which corresponds to amino acid residues 327 to 339 of the hexon-6 protein, and
CHGCGG FIGLMYY [SEQ. ID NO: 2], which corresponds to amino acid residues 333 to 339 of the hexon-6 protein containing a thioreductase motif (CxxC) within flanking residues (GG).

Dendritic cells loaded with one of these peptides are then incubated with naïve CD62L+ CD4+ cells obtained from the spleen. After several cycles of stimulation, the capacity of NKT cells to induce apoptosis of the corresponding APC is measured and a comparison is established between peptides for their capacity to induce apoptosis.

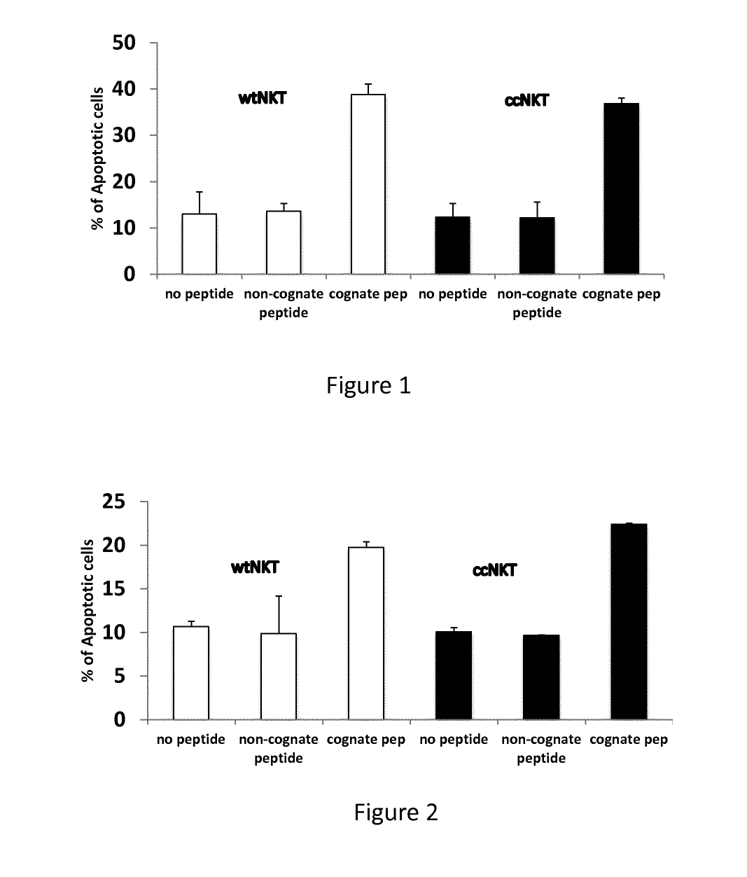

The results shown in FIG. 1 (means of 2 experiments and SEM) indicate that NKT cells expanded with peptide of either SEQ. ID NO: 1 or SEQ. ID NO: 2 elicit apoptosis of dendritic cells, as measured by annexin V binding. Negative controls include dendritic cells incubated with no peptides.

FIG. 2 shows the results (means of 2 experiments and SEM) of a similar experiments but wherein JAWS2 cells are used as APC. JAWS2 cells do not express class II-restricted molecules. The results confirm efficient induction of apoptosis of APC, confirming that recognition occurred via CD1d binding peptide presentation.

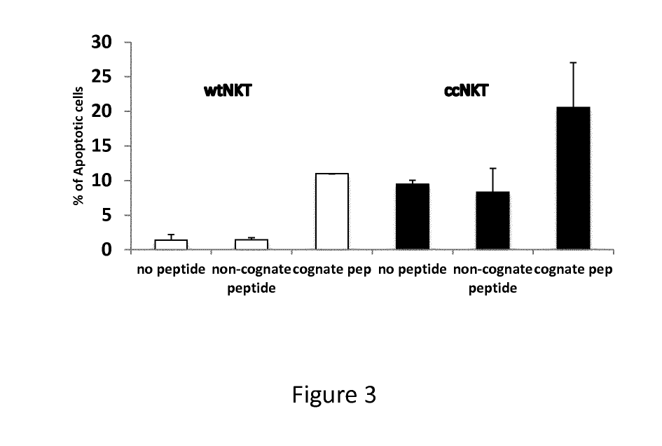

FIG. 3 depicts similar experiments (means of 2 experiments and SEM) but using a B cell line (WEHI 231 cells). WEHI cells are derived from BALB/c mice (H-2d) and not from C57BL/6 mice (H-2b). The histo-incompatibility between the 2 strains prevents presentation of peptides designed for C57BL/6 mice by BALB/c MHC class II, but not presentation by the non-polymorphic CD1d molecule. The figure shows a significant induction of apoptosis with NKT cells elicited with either peptide of SEQ. ID NO: 1 or SEQ. ID NO: 2.

Modification of the core sequence of the CD1d binding motif is carried out by introducing a hydrophobic amino acid at position P7 of the motif and generates the peptide IAFRDN FIGLMYW [SEQ. ID NO: 3], in which W at position 7 increases the binding to the hydrophobic pocket of CD1d. It is shown that this substitution further boosts the capacity of NKT cells to induce apoptosis of APCs with which a synapse is form ed.

### Example 2

### Isolation of Apoptotic Bodies

The supernatants of the apoptotic cells obtained in Example 1 are collected and submitted to two centrifugation steps (500×g, 5 min) to remove cells. The supernatants were then filtered through a 1.2 μM hydrophilic syringe filter. After centrifugation at 100,000×g for 30 minutes, apoptotic bodies contained in the pellet are harvested and used for cell experiments.

Alternatively, apoptotic cells and apoptotic bodies can be isolated by affinity using antibodies against cell surface components expressed as a result of apoptosis. An example of these are anti-thrombospondin antibodies. In a preferred preparation step, anti-thrombospondin antibodies are covalently coupled to magnetic microbeads. After incubation with gentle shaking for 1 h at 20° C., magnetic beads are retained on a magnet. Apoptotic bodies are then recovered by elution with slightly acidic buffer.

These methods are described in the prior art. See for instance Schiller et al. (2008), Cell Death Diff. 15, 183-191 and Gautier et al. (1999) J. Immunological Methods 228, 49-58.

### Example 3

### Generation of or Obtaining Immature Dendritic Cells (iDC)

Bone marrow progenitor cells are obtained from upper and lower knee bones. B and T lymphocytes are removed by magnetic depletion with CD19 and CD90 microbeads, respectively. The negative fraction containing the iDC progenitors is resuspended in serum free medium containing 500 U/ml recombinant GM-CSF and seeded (3×10⁶ cells/ml) on tissue culture plates and kept at 37° C. Cells are washed every other day for 6 days, avoiding breaking the aggregates. On day 6, iDC aggregates are removed, washed and added to a new plate. On day 7, cells are harvested and used in assays. These methods are described in the prior art. See for instance Curr. protocols Immunol., Wiley editors, vol 1, suppl. 86, 3.7.10-12.

### Example 4

### Generation of or Obtaining Antigen-Loaded Immature Dendritic Cells

iDC show a high capacity to engulf apoptotic bodies. Therefore, iDC as obtained in Example 3 are incubated with apoptotic bodies as obtained in Example 2. For this, 2×10⁵ iDC are plated in microculture wells by an incubation of 30 min at 37° C. A suspension of apoptotic bodies is then added to the culture for a further incubation of 16 h at 37° C. Cells are then washed and resuspended in medium.

### Example 5

### Use of Antigen-Loaded Dendritic Cells for Cell Therapy

iDC loaded with apoptotic bodies are injected (2×10⁵) by the intravenous route into animals prior to or after disease induction.

Thus, C57BL/6 mice are submitted to a protocol including administration of the MOG peptide (see example 1 for the peptide of SEQ. ID NO: 1) in complete Freund's adjuvant with a mycobacterium extract, and 2 injections of pertussis toxin. This protocol elicits the development of signs comparable to human multiple sclerosis within 2 weeks after MOG peptide administration.

In such a model, iDC loaded with apoptotic bodies, as obtained in Example 4, are injected to mice either one day prior to disease induction or after the first signs of disease are patent, namely 2 weeks after induction.

It is shown that, as compared to control animals in which no iDC are injected, or control animals in which unloaded iDC are injected, there is a significant prevention or suppression of disease signs.

### Example 6

### Use of Antigen-Loaded Dendritic Cells to Elicit Antigen-Specific Regulatory T Cells

iDC loaded with apoptotic bodies can be used to generate regulatory T cells in vitro.

Thus, iDC as described in Example 4 are maintained in culture.

T cells are isolated from the spleen of naïve mice by magnetic microbead sorting using antibodies to deplete CD8, CD19, CD127+ cells, followed by positive selection of CD25+ cells. The percentage of CD4+Foxp3^{high} cells is checked by fluorescence-activated cell sorting (facs) using a Foxp3 specific antibody after cell permeation. Prior art discloses the method to obtain such cells (Peters et al. (2008) Plos one 3, e3161). Cell purity above 85% is obtained.

CD4+Foxp3^{high} cells are then added (1×10⁶ cells per well) to cultures of iDCs as described in Example 4. After a stimulation cycle of 7 days at 37° C., in the presence of IL-2 (20 IU/ml), cells are washed and reincubated according to the same protocol using a fresh batch of iDC loaded with apoptotic bodies. After this second cycle of stimulation, cells can optionally be further expanded by incubation with magnetic beads coated with anti-CD3 and anti-CD28 antibodies in the presence of IL-2. Cells are then washed and evaluated by facs for expression of Foxp3.

### Example 7

### Use of Antigen-Specific Regulatory T Cells for Cell Therapy

Cells as prepared in Example 6 can be used for passive administration in the context of an autoimmune disease.

Thus, a protocol similar to the one described in Example 5 is followed but including IV administration of 2×10⁵ CD4+Foxp3^{high} cells instead of iDC.

It is shown that, as compared to control animals in which no CD4+Foxp3^{high} cells are injected, there is a significant prevention and/or suppression of disease signs.

### Example 8

### Sorting Out of Antigen-Specific Regulatory T Cells for Analytical Purposes

The population of CD4+Foxp3^{high} cells can be further analyzed to determine the importance of single component or combination of components for their mechanism of action.

Thus, CD4+Foxp3^{high} cells are separated using magnetic microbeads coated with an antibody against FasL. The two populations of cells, FasL+ and FasL(−), are then tested functionally and compared for their capacity to elicit tolerance. This is carried out using an assay system in which polyclonal effector CD4+ lymphocytes, characterized by a CD4+CD25(−) phenotype, are isolated from the spleen of a naïve animal.

Natural regulatory T cells are usually defined by their capacity to exert bystander suppression on effector cells. The assay system used here involves activation of the CD4+CD25+ T cell population by non-specific stimulation, namely a combination of anti-CD3 and anti-CD28 antibodies.

The capacity of FasL+CD4+Foxp3^{high} cells to suppress the proliferation of CD4+CD25(−) T cells is compared to that of FasL(−)CD4+Foxp3^{high} cells.

It is shown that cells expressing FasL show a higher capacity to suppress effector cell proliferation.

### Example 9

### Use of Antigen-Loaded Dendritic Cells to Elicit Antigen-Specific Regulatory NKT Cells

MHC Class II deficient iDC loaded with apoptotic bodies can be used to generate regulatory NKT cells in vitro. Thus, iDC generated as described in Example 4, but obtained from either from MHC Class II deficient mice or, alternatively, stable MHC class II negative iDC cell lines (such as JAWS2 cells), are maintained in culture.

T cells are isolated from the spleen of naïve mice by magnetic microbead sorting using antibodies to MHC class II, CD19, CD8, DX5, CD25 for depletion the cell population on antigen-presenting cells, B cells, CD8+ cells, NK cells and class-II restricted Tregs respectively.

The percentage of CD4+ NKT cells is checked by fluorescence-activated cell sorting (facs) using antibodies to NKG2D and Nkp46 and transcription factor PLZF. CD4+ cell purity above 85% is obtained.

CD4+ NKT cells are then added (1×10⁶ cells per well) to cultures of MHC Class II deficient iDCs prepared as described in Example 4. After a stimulation cycle of 7 days at 37° C., in the presence of IL-2 (100 IU/ml) and IL-15 (20 IU/ml), cells are washed and (optionally) reincubated according to the same protocol using a fresh batch of iDC loaded with apoptotic bodies. After this second cycle of stimulation, cells can optionally be further expanded by incubation with magnetic beads coated with anti-CD3 and anti-NKG2D antibodies in the presence of IL-2 (100 IU/ml) and IL-15 (20 IU/ml).

Cells are then washed and evaluated by facs for expression of surface antigens associated with regulatory activity using, for instance, antibodies to CTLA-4 and neuropilin-1.

NKT cells with regulatory function are used as in Example 7 in the context of an autoimmune disease such as multiple sclerosis, as described in Example 5.

1-17. (canceled)

18. An in vitro method of obtaining antigen-specific regulatory T cells or antigen-specific regulatory NKT cells, said method comprising the steps of:
a) providing antigen-specific NKT cells for a proteic antigen, the antigen comprising an [WFYHT]-X-X-[VILM]-X-X-[WFYHT] sequence motif of a NKT cell peptide epitope, which epitope is capable of binding to a CD1d molecule, and which antigen-specific NKT cells are obtained by contacting peripheral cells with a peptide comprising said epitope and a C-X-X-[CST] or [CST]-X-X-C redox motif;
b) providing antigen presenting cells (APCs) presenting said antigen;
c) inducing apoptosis of the APCs of b) by exposing said APCs to the antigen-specific NKT cells of a);
d) isolating apoptotic cells or apoptotic bodies from the APCs which underwent apoptosis in step c);
e) incubating said apoptotic cells or said apoptotic bodies of step d) with cells capable of presenting antigens from said apoptotic cells or from said apoptotic bodies, thereby obtaining APCs loaded with apoptotic cells or apoptotic bodies and;
f) contacting said loaded APCs obtained in step e), with a source of class II restricted CD4+ T cells, thereby obtaining a population of antigen-specific regulatory T cells, or with a source of CD1d restricted CD4+ NKT cells, thereby obtaining a population of antigen-specific regulatory NKT cells.

19. The method according to claim 18, wherein said antigen comprising said sequence motif of an NKT cell peptide epitope in step a) is:
an antigen wherein the NKT cell peptide epitope, capable of binding to a CD1d molecule, occurs in the wild type sequence of the antigen, or
an antigen wherein the NKT cell peptide epitope, capable of binding to a CD1d molecule, is generated by mutagenesis of the sequence of the antigen, or
an antigen wherein an NKT cell peptide epitope, capable of binding to a CD1d molecule, is attached to the antigen as a fusion protein.

20. The method according to claim 18, wherein sequence motif of an NKT cell peptide epitope said motif is [WF]-X-X-[IL]-X-X-[WF].

21. The method according to claim 18, wherein in step a) said antigen-specific NKT cells are obtained from naïve CD4+ T cells or from polarized CD4+ T cells.

22. The method according o claim 18, wherein in step d) said apoptotic cells or said apoptotic bodies are isolated by a method selected from the group consisting of affinity purification, centrifugation, gel filtration, magnetic beads sorting and fluorescence-activated sorting.

23. The method according to claim 18, wherein said cells capable of presenting antigens from said apoptotic cells or from said apoptotic bodies in step e) are selected from the group consisting of dendritic cells, macrophages, B lymphocytes, cells capable of expressing MHC class II determinants and cells capable of expressing CD1d determinants.

24. The method according to claim 18, wherein said cells capable of presenting antigens from said apoptotic cells or from said apoptotic bodies in step e) are selected from the group consisting of immature APCs obtainable by transformation of peripheral blood monocytes or by transformation of bone-marrow derived precursors.

25. The method according to claim 18, further comprising the step of determining the expression of Foxp3 and CD4+ in said antigen-specific regulatory T cells or said antigen-specific regulatory NKT cells.

26. The method according to claim 18, further comprising the step of separating said antigen-specific regulatory T cells into distinct subsets based on the expression of surface markers, the production of cytokines or the expression of Foxp3.

27. A population of antigen-specific regulatory NKT cells, obtainable by the method of claim 18.

28. A method of treating or preventing an autoimmune disease, an allergic disease, a graft rejection, or a chronic inflammatory disease, comprising the step of administering antigen-specific regulatory NKT cells according to claim 27 against an antigen involved in said disease.

29. The method according to claim 28, wherein said autoimmune disease is a systemic or an organ-specific autoimmune disease.

30. The method according to claim 28, wherein said autoimmune disease is against an antigen selected from the group of antigens consisting of thyroglobulin, thyroid peroxidase, TSH receptor, insulin (proinsulin), glutamic acid decarboxylase (GAD), tyrosine phosphatase IA-2, myelin oligodendrocyte protein and heat-shock protein HSP65.

31. The method according to claim 28, wherein said allergic disease is against an allergen selected from the group consisting of an airborne allergen, a food allergen, a contact allergen and a systemic allergen.

32. The method according to claim 28, wherein said graft rejection, is the rejection of a graft of cellular or of tissue origin.
